(19) United States
(12) Patent Application Publication
(10) Pub. No.: US 2021/0401404 A1
(43) Pub. Date: Dec. 30, 2021

(54) ULTRASOUND DEVICE WITH TOUCH SENSOR

(71) Applicant: Butterfly Network, Inc., Guilford, CT (US)

(72) Inventors: Matthew R. Hageman, Winfield, IL (US); Adam Mekeel Mack, Half Moon Bay, CA (US)

(73) Assignee: Butterfly Network, Inc., Guilford, CT (US)

(21) Appl. No.: 17/344,794

(22) Filed: Jun. 10, 2021

Related U.S. Application Data
(60) Provisional application No. 63/046,381, filed on Jun. 30, 2020.

(51) Int. Cl. A61B 8/00; A61B 8/08

(52) U.S. Cl. CPC A61B 8/467; A61B 8/461; A61B 8/5207

(57) ABSTRACT

Aspects of the technology described herein relate to detection of one or more touches by an ultrasound device. An ultrasound device may include touch detection circuitry and may be configured to detect, with the touch detection circuitry, one or more touches on an exterior touch surface of the ultrasound device during ultrasound imaging, and to transmit an indication of detection of the one or more touches to a processing device in operative communication with the ultrasound device during the ultrasound imaging. The processing device may be configured to, in response to receipt of the indication from the ultrasound device, perform an action to control an aspect of the ultrasound imaging based on information included in the received indication. The touch detection circuitry may include capacitive sensing circuitry and/or resistive sensing circuitry to detect a touch made with little or no force.

### CROSS-REFERENCE TO RELATED APPLICATIONS

The present application claims the benefit of U.S. Provisional Application No. 63/046,381 filed Jun. 30, 2020 under Attorney Docket No. B1348.70189US00, and entitled “ULTRASOUND DEVICE WITH TOUCH SENSOR”, which is hereby incorporated by reference herein in its entirety.

### FIELD

Generally, aspects of the technology described herein relate to ultrasound devices. Some aspects relate to detection of one or more touches on an ultrasound device itself, with the detection being made by the ultrasound device itself.

### BACKGROUND

Ultrasound devices may be used to perform diagnostic imaging and/or treatment, using sound waves with frequencies that are higher than those audible to humans. Ultrasound imaging may be used to see internal soft tissue body structures. When pulses of ultrasonic sound waves are transmitted into tissue by a probe, sound waves of different amplitudes may be reflected back towards the probe at different tissue interfaces. These reflected sound waves may then be recorded and displayed as an ultrasound image to an operator of the probe. The strength (e.g., amplitude) of a sound signal of the reflected sound waves and the time it takes for the sounds waves to travel through the body may provide information used to produce the ultrasound image. Many different types of images can be formed using ultrasound devices. For example, images can be generated that show two-dimensional cross-sections of tissue, blood flow, motion of tissue over time, the location of blood, the presence of specific molecules, the stiffness of tissue, and the anatomy of a three-dimensional region.

### SUMMARY

According to one aspect of the technology of the present application, an ultrasound device is provided. The ultrasound device may comprise a housing and touch detection circuitry housed at least partially in the housing. The touch detection circuitry may be configured to detect at least one touch of a user on a circumferential region of the housing during ultrasound imaging, and to transmit an indication of detection of the at least one touch to a processing device during the ultrasound imaging. The circumferential region may partially or completely encircle an exterior portion of the housing.

In some embodiments of this aspect, the touch detection circuitry may comprise a touch-sensitive surface on the circumferential region of the housing. In some embodiments, the touch-sensitive surface may be a ring-shaped surface encircling a portion of the housing between a shroud portion that connects a lens portion, through which ultrasonic waves are transmitted and received, and a handle portion configured to be held in a hand of the user. In some embodiments, the touch-sensitive surface may comprise an exterior surface of the shroud portion.

In some embodiments of this aspect, the touch detection circuitry may comprise resistive sensing circuitry for at least one resistive sensor. The resistive sensing circuitry may be configured to detect the at least one touch by detecting a change in resistance of at least one part of the circumferential region of the housing. In some embodiments, the change in resistance may be a change from a resistive state when the circumferential region of the housing is not being touched to a short-circuited state when the circumferential region of the housing is being touched.

In some embodiments of this aspect, the touch detection circuitry may comprise capacitive sensing circuitry for at least one capacitive sensor. The capacitive sensing circuitry may be configured to detect the at least one touch by detecting a change in capacitance of at least one part of the circumferential region of the housing.

In some embodiments of this aspect, the touch detection circuitry may be configured to detect simultaneous touches at a plurality of parts of the circumferential region of the housing.

In some embodiments of this aspect, the touch detection circuitry may be configured to detect consecutive touches on the circumferential region of the housing.

In some embodiments of this aspect, the touch detection circuitry may be configured to detect at least one touch of a user on the circumferential region of the housing before the ultrasound imaging starts and after the ultrasound imaging ends, and to transmit an indication of detection of the at least one touch to the processing device before the ultrasound imaging starts and after the ultrasound imaging ends.

In some embodiments of this aspect, the ultrasound device may further comprise the processing device. The processing device may be external to the housing or may be housed at least partially in the housing. In some embodiments, the processing device may be configured to receive from the touch detection circuitry the indication of detection of the at least one touch, and to perform an action to control an aspect of the ultrasound imaging based on the at least one touch. When performing the action to control the aspect of the ultrasound imaging, the processor may control any one or any combination of: to record a cine, to freeze a current ultrasound image on a display screen of the processing device, to save to memory an ultrasound image that is frozen on the display screen, to modify an imaging depth, to modify a gain, to toggle color Doppler imaging on or off, to switch imaging modes, and to switch imaging presets.

According to another aspect of the technology of the present application, a method is provided, relating to use of an ultrasound device. The method may comprise: detecting, using touch detection circuitry of the ultrasound device, at least one touch of a user on a circumferential region of the ultrasound device during ultrasound imaging; and transmitting an indication of detection of the at least one touch to a processing device during the ultrasound imaging. The circumferential region may partially or completely encircle an exterior portion of the ultrasound device

In some embodiments of this aspect, the detecting may detect the at least one touch on a ring-shaped surface encircling a portion of the ultrasound device.

In some embodiments of this aspect, the detecting may detect the at least one touch on a shroud surface of the ultrasound device.

In some embodiments of this aspect, the detecting may detect the at least one touch by detecting a change in resistance of at least one part of the circumferential region. In some embodiments, the change in resistance is a change from a resistive state, when the circumferential region is not being touched, to a short-circuited state, when the at least one part of the circumferential region is being touched.

In some embodiments of this aspect, the detecting may detect the at least one touch by detecting a change in capacitance of at least one part of the circumferential region.

In some embodiments of this aspect, the transmitting of the indication of detection of the at least one touch to the processing device may cause the processing device to perform an action to control any one or any combination of: to record a cine, to freeze a current ultrasound image on a display screen of the processing device, to save to memory an ultrasound image that is frozen on the display screen, to modify an imaging depth, to modify a gain, to toggle color Doppler imaging on or off, to switch imaging modes, and to switch imaging presets.

The foregoing and other aspects, embodiments, and features disclosed in the present application can be more fully understood from the following description in conjunction with the accompanying drawings.

### BRIEF DESCRIPTION OF THE DRAWINGS

The skilled artisan will understand that the following figures, described herein, are for illustration purposes only. It is to be understood that in some instances various aspects of the technology described herein may be shown exaggerated or enlarged to facilitate an understanding of the technology. In the drawings, like reference characters may be used to refer to like features, which may be functionally similar and/or structurally similar elements, throughout the various figures. The drawings are not necessarily to scale, as emphasis is instead placed on illustrating and teaching principles of the various aspects of the technology. The drawings are not intended to limit the scope of the technology taught herein in any way.

FIG. 1 shows a schematic block diagram of an example ultrasound system, in accordance with certain embodiments of the technology described herein;

FIG. 2 shows a non-limiting example of an ultrasound device, in accordance with certain embodiments described herein;

FIG. 3 shows another non-limiting example of an ultrasound device, in accordance with certain embodiments described herein;

FIG. 4A shows an elevational view of a head region of an ultrasound device, in accordance with certain embodiments described herein;

FIG. 4B shows a perspective view of the head region of FIG. 4A in a partially disassembled state;

FIG. 4C shows a cross section A-A of the head region of FIG. 4A;

FIG. 4D shows an enlargement of a region of FIG. 4C;

FIG. 4E shows a schematic diagram illustrating how an ultrasound device may be touched to provide user input, in accordance with certain embodiments described herein;

FIG. 5A shows an elevational view of a head region of an ultrasound device, in accordance with certain embodiments described herein;

FIG. 5B shows a cross section C-C of the head region of FIG. 5A;

FIG. 5C shows an enlargement of a region of FIG. 5B;

FIG. 6A shows a perspective view of a head region of an ultrasound device in a partially disassembled state, in accordance with certain embodiments described herein;

FIG. 6B shows a cross-sectional view of a portion of a flexible outer ring of the head region of FIG. 6A;

FIG. 6C shows a plan view of a section of a shorting strip of the head region of FIG. 6A;

FIG. 6D shows a cross-sectional view of the shorting strip of FIG. 6C shorted by an inner layer of the flexible outer ring of FIG. 6B under weight of a finger;

FIG. 7 shows a process for detecting one or more touches on an ultrasound device, in accordance with certain embodiments described herein;

FIG. 8 shows a process for performing one or more actions based on a detection of one or more touches on an ultrasound device, in accordance with certain embodiments described herein; and

FIG. 9 shows a schematic diagram illustrating how an ultrasound device may be used to image a subject, in accordance with certain embodiments described herein.

### DETAILED DESCRIPTION

Certain embodiments of ultrasound systems include an ultrasound device (e.g., a handheld ultrasound probe) and a processing device in operative communication with the ultrasound device (e.g., across one or both of a wired communication link and a wireless communication link). The processing device may be a portable device, (e.g., a phone, tablet, a laptop) and may be configured to display on a display screen a graphical user interface (“GUI” herein) through which an operator or user of the ultrasound device may view recently collected ultrasound images and control aspects of ultrasound imaging. For example, a user may control any one or any combination of: recording of a cine (i.e., a sequence of ultrasound images); freezing of a current ultrasound image on the display screen; saving to memory the ultrasound image that is frozen on the display screen; modification of an imaging depth; modification of a gain; on/off toggling of a color Doppler imaging function; switching of imaging modes (e.g., biplane, spectra Doppler, power Doppler imaging modes); and switching of imaging presets (e.g., sets of imaging parameters optimized for imaging particular anatomies).

In performing ultrasound imaging on a patient, it may be helpful in some instances to enable a user to control aspects of the ultrasound imaging (e.g., the aspects listed above, as well as others) without needing to interact with a GUI displayed on the display screen of the processing device. For example, touching the display screen of the processing device may interfere with or compromise the user's sterility, which may be a particular concern when the ultrasound imaging is being performed on, e.g., a patient with an impaired immune system. In another example, a user may have both hands occupied (e.g., one hand holding the ultrasound device and another hand holding an instrument such as a needle) and thus may not have a free hand to touch the display screen. In another example, a user may be wearing gloves on his/her hands, and the processing device may not register touches to the display screen by gloved hands. In some cases, removing gloves in order to interact with a GUI may be undesirable, because it may be advantageous and desirable for the user to execute a current clinical procedure as swiftly as possible. Particularly in anesthesia and interventional radiology, reduced procedure time may be associated with reduced infection and complication risk as well as increased throughput.

The inventors have recognized that including touch detection circuitry in an ultrasound device (e.g., a handheld ultrasound probe) may enable the ultrasound device to detect one or more touches on an exterior part of the ultrasound device. For example, as described below, the exterior part may be a diffuse region that may encompass a significant surface area of the ultrasound device (e.g., an entirety or a majority of a shroud of the ultrasound device), or may be a plurality of regions that may be diffusely spread out on the ultrasound device (e.g., a plurality of discrete touch surfaces, which may be arranged at various locations for easy touching). The touch detection circuitry in the ultrasound device may be configured to detect one or more touches on the exterior part of the ultrasound device during ultrasound imaging without an adverse impact on the ultrasound imaging. A touch need not be forceful in order to be detected, and a gentle touch contact on the exterior part may be sufficient. By enabling a gentle touch to be detected, the touch detection circuitry may avoid concerns about bumps, vibrations, shakes, etc., having a negative impact on the ability to obtain clear and reliable still and/or video ultrasound images. Aspects of the present technology described herein provide an ultrasound device with touch detection circuitry that may be configured to detect one or more touches after an ultrasound imaging session has begun, after the ultrasound device has begun to collect ultrasound data, and/or while the ultrasound device is placed on the subject for ultrasound imaging, as well as before and/or after the imaging session. As described below, the touch detection circuitry may be configured to detect touches with sufficient sensitivity by detecting a change in an electrical property at one or more points of contact (i.e., touch points) on the exterior part of the ultrasound device. The touches detected by the touch detection circuitry may be sufficiently gentle that imaging is not adversely impacted.

In response to detecting one or more touches on the exterior part of the ultrasound device, the ultrasound device may be configured to transmit an indication of this detection to the processing device during the ultrasound imaging. In response to receiving this indication, the processing device may be configured to perform an action related to controlling an aspect of ultrasound imaging, during the imaging itself. The touch detection circuitry may be configured to detect one or more touches (“touch(es)” herein) with sufficient sensitivity such that touch(es) made with little to no force may be detectable, so as not to interfere with the ultrasound device's imaging. The action or actions performed by the processing device in response to the touch(es) may control various aspects of an ultrasound imaging process, and not just initiation of ultrasound imaging. For example, an action controlled via a touch of a user of the ultrasound device may be to initiate or to stop recording of a cine, to freeze a current ultrasound image on the display screen of the processing device, to save to memory the ultrasound image that is frozen on the display screen, to modify an imaging depth, to modify a gain, to toggle on or off a color Doppler imaging function, to switch imaging modes, to switch imaging presets, or any combination of these actions. In some embodiments of the present technology, in performing an action controlled via a touch of a user of the ultrasound device, the processing device may modify its own configuration. In some embodiments of the present technology, in performing an action controlled via a touch of a user of the ultrasound device, the processing device may modify a configuration of the ultrasound device (e.g., by transmitting a command to the ultrasound device). In some embodiments of the present technology, in performing an action controlled via a touch of a user of the ultrasound device, the processing device may modify both a configuration of the processing device itself and a configuration of the ultrasound device. Thus, a user may be able to control certain aspects of ultrasound imaging by touching the exterior part of the ultrasound device, and without needing to touch the display screen of the processing device. Using touch(es) on the exterior part of the ultrasound device, rather than, e.g., pressing an exterior button on the ultrasound device, to control aspects of ultrasound imaging, may advantageously prevent leakage of fluids into a housing of the ultrasound device, because interfaces or seams around an exterior button may provide possible access points for liquid to seep into an interior of the housing of the ultrasound device.

It should be appreciated that the embodiments described herein may be implemented in any of numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect.

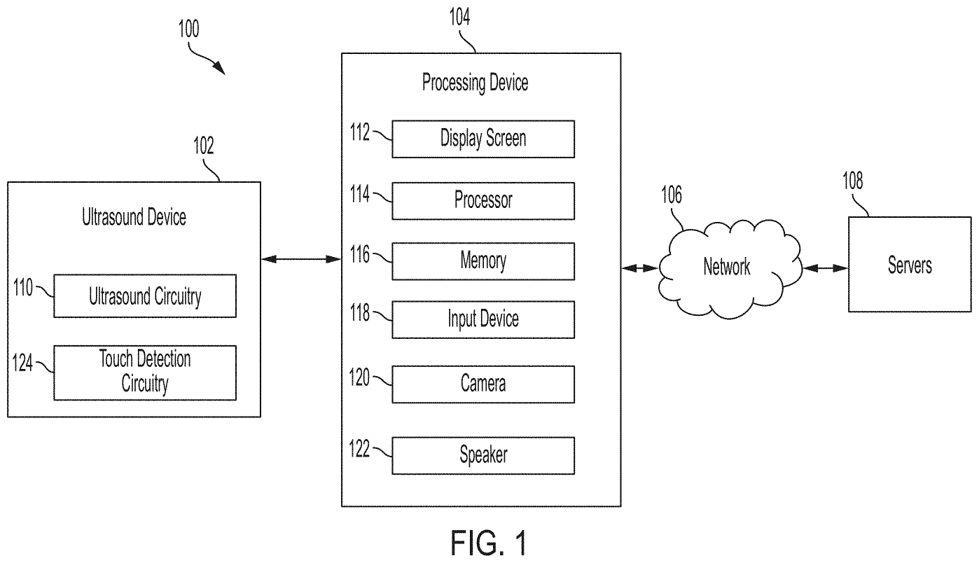

FIG. 1 illustrates a schematic block diagram of an example ultrasound system 100, in accordance with certain embodiments of the technology described herein. The ultrasound system 100 may include an ultrasound device 102 and a processing device 104.

The ultrasound device 102 may include ultrasound circuitry 110 and touch detection circuitry 124. The processing device 104 may include a display screen 112, a processor 114, a memory 116, an input device 118, a camera 120, and a speaker 122. The processing device 104 may be in wired communication (e.g., through a lightning connector or a mini-USB connector) and/or wireless communication (e.g., using BLUETOOTH, ZIGBEE, and/or WiFi wireless protocols) with the ultrasound device 102.

Although the processing device 104 is shown in FIG. 1 to be separate from the processing device 104, in some embodiments of the present technology one or more features of the processing device 104 may be incorporated in the ultrasound device 102. For example, in addition to (or instead of) the processor 114 and the memory 116 of the processing device 104, the ultrasound system 100 may include a processor and/or a memory in the ultrasound device 102, to perform some or all of the functions of the processor 114 and/or the memory 116 on board the ultrasound device 102. In another example, the ultrasound system 100 may include a display screen in the ultrasound device 102, which may be smaller than the display screen 112 of the processing device 104 but which may provide a user with, e.g., general information sufficient to confirm that a region of interest of a patient is being imaged, so that the user does not need to look away from the ultrasound device 102 to see an image on the display screen 112 of the processing device 104.

The ultrasound device 102 may be configured to generate ultrasound data that may be employed to generate an ultrasound image. The ultrasound device 102 may be constructed in any of a variety of ways. In some embodiments, the ultrasound device 102 may include a transmitter that transmits a signal to a transmit beamformer, which in turn drives transducer elements within a transducer array to emit pulsed ultrasonic signals (i.e., pulses of ultrasonic waves) into an object, such as a patient. The pulsed ultrasonic signals may be back-scattered from structures in the patient, such as blood cells, muscular tissue, bone, and the like, to produce echoes that return to the transducer elements. These echoes may then be converted into electrical signals by the transducer elements. These electrical signals, which represent received echoes of the pulsed ultrasonic signals back-scattered from the structures in the patient, are sent to a receive beamformer that outputs ultrasound data. The ultrasound circuitry 110 may be configured to generate the ultrasound data. The ultrasound circuitry 110 may include one or more ultrasonic transducers monolithically integrated onto a single semiconductor die. The ultrasonic transducers may include, for example, one or more capacitive micromachined ultrasonic transducers (CMUTs), one or more CMOS (complementary metal-oxide-semiconductor) ultrasonic transducers (CUTs), one or more piezoelectric micromachined ultrasonic transducers (PMUTs), and/or one or more other suitable ultrasonic transducer cells. In some embodiments of the present technology, the ultrasonic transducers may be formed on the same chip as other electronic components in the ultrasound circuitry 110 (e.g., transmit circuitry, receive circuitry, control circuitry, power management circuitry, and processing circuitry) to form a monolithic ultrasound device. The ultrasound device 102 may transmit ultrasound data and/or ultrasound images to the processing device 104 over a wired communication link (e.g., through a lightning connector or a mini-USB connector) and/or a wireless communication link (e.g., via BLUETOOTH, ZIGBEE, and/or WiFi wireless protocols).

The touch detection circuitry 124 may be configured to detect a touch (or multiple touches) on an exterior touch surface of the ultrasound device 102. In some embodiments of the present technology, the touch detection circuitry 124 may include capacitive sensing circuitry, which may include processing circuitry to process capacitance data from the capacitive sensing circuitry and detect touch(es) based on the capacitance data. In some embodiments of the present technology, the touch detection circuitry 124 may include resistive sensing circuitry, which may include processing circuitry to process resistance data from the resistive sensing circuitry and detect touch(es) based on the resistance data. Capacitive sensing and resistive sensing are described in more detail below. In some embodiments, the exterior touch surface may comprise a diffuse region of the ultrasound device 102. In one example, the exterior touch surface may encompass a wide surface area, such as an entirety or a majority (greater than 50%) of a surface of a shroud of the ultrasound device 102. In another example, the exterior touch surface may include a circumferential region that partially or entirely encircles part of the ultrasound device 102, such as a ring that forms a 360° circumferential band around part of the ultrasound device 102 or a partial ring that forms a circumferential band encircling less than 360° (e.g., a circumferential band that encircles about 90° or greater (˜25% or more) of the ultrasound device 102; or about 180° or greater (˜50% or more); or about 270° or greater (˜75% or more)). In some embodiments, the exterior touch surface may comprise a plurality of touch regions that may be discrete from each other and positioned at various locations for easy touching on the ultrasound device 102. In one example, the plurality of touch regions may be arranged circumferentially on the ultrasound device 102 at discrete locations along a circumferential band or a plurality of circumferential bands, with the touch regions being formed of separate individual touch-sensing circuits.

It should be understood that the term “touch,” as used herein, is a type of a contact that may involve little to no acceleration of a touching object onto a touch surface in order to be detected. In contrast, a “tap” type of contact involves at least a minimal threshold amount of acceleration (e.g., at least 100 mg, where g is Earth's gravitational acceleration) in order to be detected. Thus, it should be understood that touch contact and tap contact are different types of contacts. This difference may be understood with reference to a typical touch pad on, e.g., a laptop computer. With a touch pad, gentle glide of a finger across the touch pad's surface is sufficient to be detected and to move a displayed cursor on a screen of the laptop computer. In order to input a selection, a user typically can tap on the touch pad's surface. However, the user must make contact with the touch pad's surface with at least a minimum amount of acceleration in order for the contact to be detected as a tap for a selection input. When there is insufficient acceleration, the user's contact is not registered or detected as a selection input.

In some embodiments of the present technology, the touch detection circuitry 124 may be configured to detect and differentiate between a single touch (e.g., an individual touch by a finger), multiple touches (e.g., simultaneous touches by two or more fingers), and a series of touches (e.g., two or more single touches performed sequentially, two or more multiple touches performed sequentially).

In some embodiments of the present technology, to detect a touch, the touch detection circuitry 124 may be configured to determine whether a measured capacitance has changed by at least a predetermined value. In one example, the touch detection circuitry 124 may determine that a touch has occurred when a currently measured capacitance changes by greater than a predetermined amount from the current capacitance. In another example, the touch detection circuitry 124 may determine that a touch has occurred when a measured capacitance has exceeded a predetermined threshold value. As will be appreciated, other criterion or criteria may be used to determine when a change in capacitance is to be detected as a touch.

As mentioned above, the processing device 104 may control various aspects of an ultrasound imaging process in response to detected touch(es). In some embodiments of the present technology, detection of a single touch may control a first aspect of the ultrasound imaging process, detection of two consecutive touches may control a second aspect, detection of three consecutive touches may control a third aspect, detection of two simultaneous touch may control a fourth aspect, and so on.

In some embodiments of the present technology, if a detected touch lasts for greater than a predetermined amount of time (e.g., 2 seconds, or 3 seconds, or 4 seconds, or 5 seconds), or if a detected touch is momentary and no additional touch is detected in the predetermined amount of time, then the processing device 104 may determine that a single touch was made, and may control the ultrasound imaging process accordingly (e.g., control a first aspect of the imaging process). A touch may be determined to be momentary if a measured capacitance, e.g., rises to satisfy capacitance-change criterion or criteria to be detected as a touch and then, within the predetermined amount of time (e.g., 2 seconds, or 3 seconds, or 4 seconds, or 5 seconds) falls so that the criterion or criteria no longer are satisfied. A momentary touch followed by another touch within the predetermined period of time may be determined by the processing device 104 to be a double touch and may result in the processing device 104 controlling, e.g., a second aspect of the imaging process. Similarly, if the touch detection circuitry 124 detects three touches within the predetermined amount of time, the processing device 104 may control a fourth aspect of the imaging process. As described below, the touch detection circuitry 124 may detect simultaneous touches, i.e., concurrent touches at different locations on the touch surface, via a plurality of touch circuits. When two or more touch circuits of the touch detection circuitry 124 satisfy the capacitance-change criterion or criteria at the same time, the processing device 104 may control a fourth aspect of the imaging process.

The touch detection circuitry 124 may be configured to detect touch(es) on an exterior surface of the ultrasound device 102 during ultrasound imaging. More specifically, the touch detection circuitry 124 may be configured to detect touch(es) after an ultrasound imaging session has begun on a subject (e.g., a patient), after the ultrasound device 102 has begun to collect ultrasound data (e.g., back-scattered ultrasonic waves), and/or while the ultrasound device 102 is placed on the subject for ultrasound imaging. The touch detection circuitry 124 also may be configured to detect touches(es) before an imaging session has begun and/or after the imaging session is over (e.g., to change an imaging mode before the ultrasound device 102 is placed on the subject and/or after the ultrasound device 102 is moved away from the subject).

The touch detection circuitry 124 may be configured to detect touch(es) made with little or no force on the touch surface of the ultrasound device 102, and thus would not interfere with imaging being performed by the ultrasound device 102. That is, touch(es) may be detected in which little or no force is applied (e.g., less than about 75 mg, less than about 50 mg, less than about 25 mg, or substantially 0 mg). For example, a finger or fingers already touching an external surface of the ultrasound device 102 may be gently slid onto the touch surface without application of pressure, or with only minimal pressure being applied (e.g., merely the weight of a finger would be sufficient).

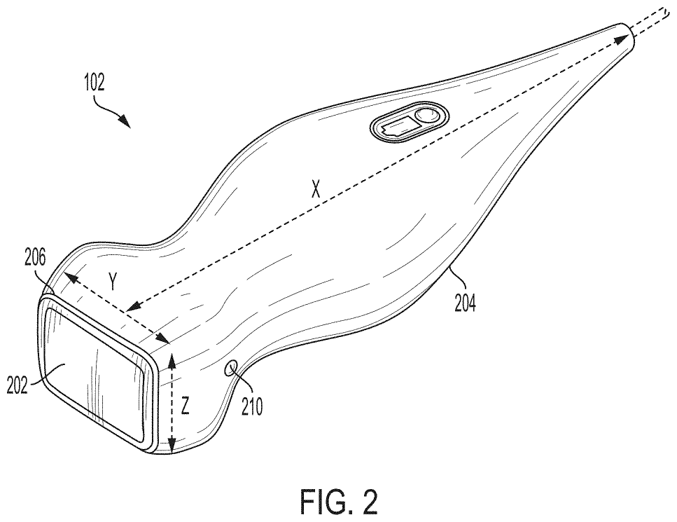

The touch surface of the touch detection circuitry 124 may be configured on the ultrasound device 102 so that it may be easily touched by a user (e.g., an ultrasound technician at a medical clinic). That is, the user may use a single hand to hold the ultrasound device 102 and also to touch the touch surface on the ultrasound device 102 while holding the ultrasound device 102. FIG. 2 illustrates an embodiment in which the ultrasound device 102 may be described according to three axes (x, y, and z), as shown. The ultrasound device 102 may comprise a housing 204 that is elongated along the x-axis and that may be contoured to be easily grasped by the user's hand. The housing 204 may have an interior portion in which the ultrasound circuitry 110 and the touch detection circuitry 124 are housed. The ultrasound circuitry 110 may be configured to produce ultrasonic waves that are emitted through a lens 202 having an exterior surface that is generally perpendicular to the x-axis. For example, the exterior surface of the lens 202 may extend generally along an y-z plane and may have a slightly convex shape to, e.g., maximize skin contact with a patient. Ultrasonic waves back-scattered from internal portions of the patient may be received by the ultrasound circuitry 110 through the lens 202. The lens 202 may be held in place on the housing 204 by a shroud 206. In some embodiments of the present technology, through use of liquid-impervious housing materials and seals, the ultrasound device 102 may have a leak-tight construction such that liquids (e.g., ultrasound gels, water, etc.) may be prevented from entering the interior portion of the housing 204. For example, interfaces between the lens 202 and the shroud 206 and the housing 204, as well as other interfaces, may be sealed against liquid penetration via, e.g., a rubber gasket or the like.

In some embodiments of the present technology, the touch surface may comprise a surface spot 210 on the ultrasound device 102. The surface spot 210 may be located at a position that may be easily touched by a hand holding the ultrasound device 102 by, e.g., sliding a thumb of the hand onto the surface spot 210. The surface spot 210 may be formed of a non-conductive material and may be surrounded by grounded non-conductive material of the housing 204. The non-conductive material of the surface spot 210 may serve as a dielectric portion of a capacitor of a capacitive sensor (not shown) of the touch detection circuitry 124. The capacitive sensor may include an inner conductor, which may be a conductive layer located on an interior side of the non-conductive material, with the surface spot 210 corresponding to an exterior side of the non-conductive material opposite the interior side. The inner conductor may be electrically connected to sensing circuitry of the capacitive sensor, which may, e.g., apply a known charge to the inner conductor when the ultrasound device 102 is powered on or when the sensing circuitry is activated after the ultrasound device 102 is powered on. In such a state, when the surface spot 210 is not being touched by a finger, the sensing circuitry of the capacitive sensor may measure a parasitic capacitance. However, when a finger of the user touches the surface spot, the finger may have sufficient conductivity to serve as an outer conductor of the capacitor and cause a change in capacitance to be measured by the sensing circuitry. The sensing circuitry may be sufficiently sensitive that a touch by a gloved finger may have sufficient conductivity to cause a measurable change in capacitance. For example, a detectable change in capacitance may be reliably detected when the surface spot 210 is touched by a finger wearing a standard 3-mil-thick nitrile glove.

As will be appreciated, although a single surface spot 210 is shown in FIG. 2, in some embodiments of the present technology the ultrasound device 102 may comprise a plurality of such surface spots 210 diffusely located at positions that may be conveniently touched by more than one of the user's fingers. Each surface spot 210 may form its own capacitor, and the sensing circuitry may be configured to measure a capacitance of each surface spot 210 individually and to differentiate a capacitance from one surface spot 210 from a capacitance of another surface spot 210. Thus, the sensing circuitry of the touch detection circuitry 124 may be able to detect and differentiate between a touch of a finger on one surface spot 210 (e.g., a touch by an index finger of the user) and simultaneous touches on multiple surface spots 210 (e.g., concurrent touches by the user's index and middle fingers). In some embodiments, an array of surface spots 210 may be arranged at various locations relative to a circumference line encircling the ultrasound device 102. The circumference line may be, for example, on a plane perpendicular to the x-axis or on a plane inclined relative to the x-axis. In some embodiments, the ultrasound device 102 may have surfaces that can be designated as top, bottom, left, and right (e.g., labeling or other indicia may indicate the top surface). Multiple surface spots 210 may be arranged at various locations, e.g., on one side, or on two adjacent sides, or on two opposing sides, or on three sides, or on all sides. In some embodiments, multiple touches may be detected simultaneously on opposite sides of the ultrasound device 102 and/or on adjacent sides of the ultrasound device.

In some embodiments of the present technology, the region of the ultrasonic device 102 that is most easily touched by one or more of the user's fingers is a head region, which may encompass the shroud 206 and a portion of the housing 204 close to the shroud 206. As can be seen from FIG. 2, if the user holds the ultrasound device 102 such that the lens 202 is on a patient and a palm of the user's hand is at or near a central region along the x-axis, the user's fingers would extend generally toward the head region. Thus, in some embodiments, the surface spot(s) 210 may be advantageously positioned in the head region within reaching distance of the user's fingers.

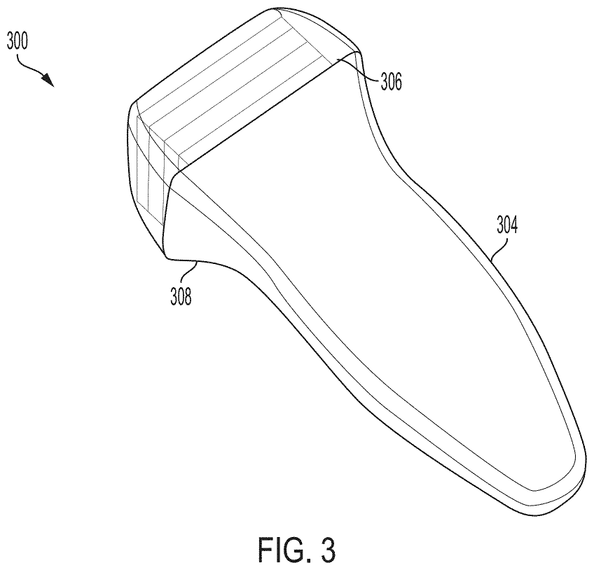

FIG. 3 shows a perspective view of an example of an ultrasonic device 300 according to various embodiments of the present technology. The ultrasonic device 300 may comprise a housing 304, which may serve as a handle that is held in a user's hand, and a shroud 306 configured to hold a lens in place relative to the housing 304. In this perspective view, the lens of the ultrasonic device 300 cannot be seen. In contrast to the shroud 206 of the ultrasonic device 102 of FIG. 2, the shroud 306 has a greater surface area. The ultrasonic device 300 may have a head region that includes the shroud 306 and a portion 308 of the housing 304 near the shroud 306. As discussed above, in some embodiments of the present technology, one or more touch surface(s) may advantageously be positioned in the head region.

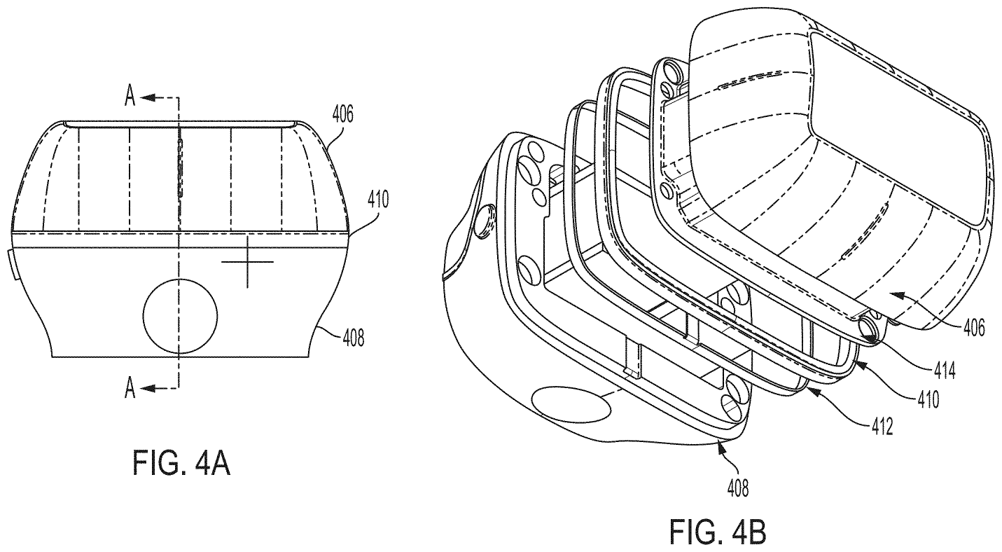

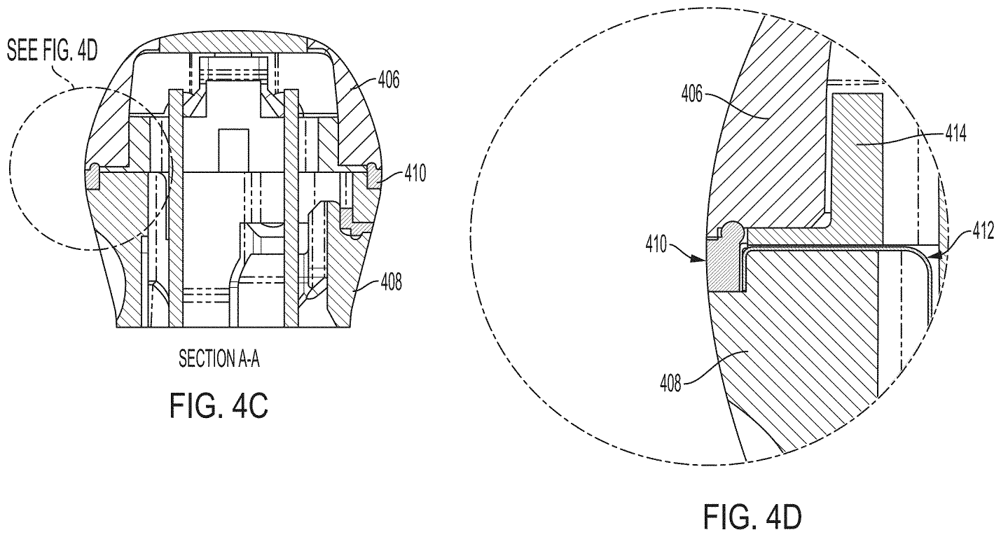

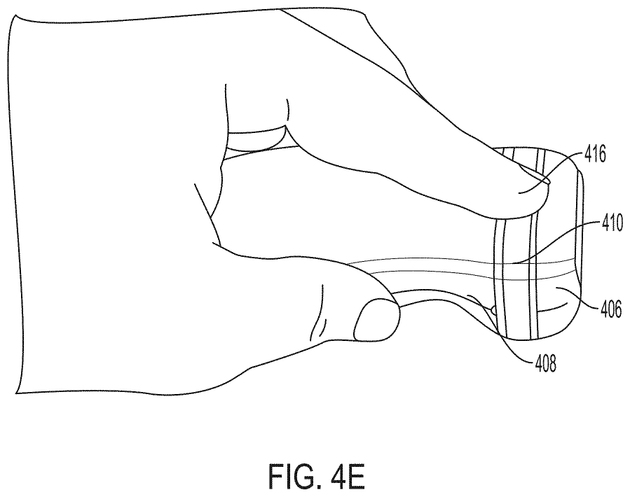

FIG. 4A shows an elevational view of a head region of an ultrasound device, according to some embodiments of the present technology. FIG. 4B shows a perspective view of the head region of FIG. 4A, in a partially disassembled state, FIG. 4C shows a cross section A-A of the head region of FIG. 4A, and FIG. 4D shows an enlargement of a region of FIG. 4C. The head region includes a shroud 406 attached to a housing portion 408 via a gasket 410. The shroud 406 may be formed of a non-conductive material (e.g., hard plastic) and may be electrically grounded. The housing portion 408 may be formed of a non-conductive material (e.g., hard plastic) and may be grounded. The shroud 406 may be supported by a shroud adapter 414, which may be contoured to support at least one internal surface of the shroud 406, as depicted in FIG. 4D. The shroud adapter 414 may be formed of a hard material, conductive or non-conductive, and may be electrically grounded. The gasket 410 may be formed of a non-conductive material and may be electrically floating. In some embodiments of the present technology, the gasket 410 may be a circumferential ring or loop formed of an elastomer (e.g., rubber) encircling a circumference of the ultrasound device, and may provide a liquid-impervious seal between the shroud 406 and the housing portion 408. An exterior surface of the gasket 410 may be flush with adjacent exterior surfaces of the shroud 406 and the housing portion 408, as shown in FIG. 4D, or, for easy touchability, may be raised relative to the adjacent exterior surfaces of the shroud 406 and the housing portion 408.

In some embodiments of the present technology, the gasket 410 may form part of a capacitive touch detector. More specifically, the gasket 410 may form a ring-shaped dielectric portion of a capacitor of the capacitive touch detector. A conductive strip 412 may have a ring-shaped portion positioned adjacent an internal surface of the gasket 410 and may serve as an inner conductor of the capacitor. The conductive strip 412 may be electrically connected to sensing circuitry of the capacitive touch sensor, and the sensing circuitry may, e.g., apply a known charge (e.g., a known voltage) to the conductive strip 412 when the ultrasound device is powered on. In some embodiments, a portion of the conductive strip 412 may be electrically connected to circuitry on a printed circuit board or other structure in the housing.

Similar to the description above, when the exterior surface of the gasket 410 is not being touched by a finger, the sensing circuitry of the capacitive touch sensor may measure a parasitic capacitance, which may be used as a baseline capacitance. However, when the user's finger(s) touch(es) the exterior surface of the gasket 410, the finger(s) may have sufficient conductivity to serve as an outer conductor of the capacitor and cause a change in capacitance to be measured by the sensing circuitry. The sensing circuitry may be sufficiently sensitive that a touch by a gloved finger may have sufficient conductivity to cause a measurable change in capacitance.

An advantageous aspect of the ring-shaped structure of the gasket 410 and the conductive strip 412 is that the structure enables a touch as well as consecutive touches to be detectable at any location on the circumferential exterior surface of the gasket 410, thus allowing the user to use any available finger(s) to provide touch input to the processing device 104. Another advantageous aspect of the ring-shaped structure of the gasket 410 is that it may also provide a leak-tight seal that is impervious to liquids, as noted above. FIG. 4E shows a schematic diagram illustrating how a finger 416 of the user may touch the exterior surface of the gasket 410, leaving other portions of the user's hand free to hold the ultrasound device and/or perform other tasks. The finger 416 may touch the exterior surface of the gasket 410 without imparting any deliberate force by, e.g., sliding the finger 416 from a non-touch-sensitive region, such as the housing portion 408 in some embodiments of the present technology, onto the exterior surface of the gasket 410, which may detect touch(es) via a measured change in capacitance, as discussed above.

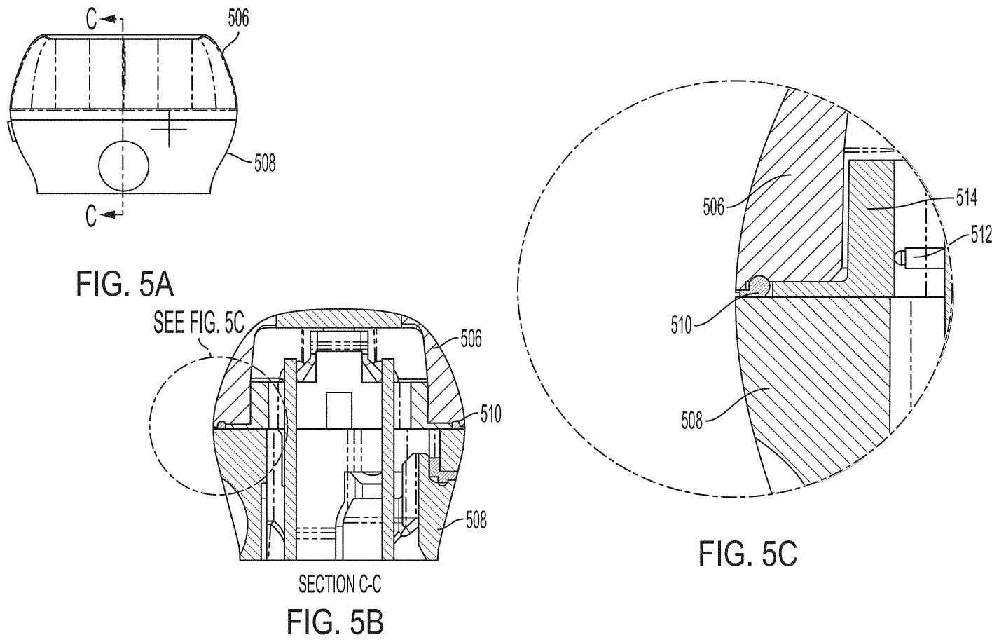

FIG. 5A shows an elevational view of a head region of an ultrasound device, according to some embodiments of the present technology. FIG. 5B shows a cross section C-C of the head region of FIG. 5A, and FIG. 5C shows an enlargement of a region of FIG. 5B. The head region includes a shroud 506 attached to a housing portion 508 via a gasket 510. The shroud 506 may be formed of a non-conductive material (e.g., hard plastic) and may be electrically floating. The housing portion 508 may be formed of a non-conductive material (e.g., hard plastic) and may be grounded. The shroud 506 may be supported by a shroud adapter 514, which may be contoured to support at least one internal surface of the shroud 506, as depicted in FIG. 5C. The gasket 510 may be formed of a non-conductive material and may be electrically floating. In some embodiments of the present technology, the gasket 510 may be a ring or loop formed of an elastomer (e.g., rubber), which may provide a liquid-impervious seal between the shroud 506 and the housing portion 508. An exterior surface of the gasket 510 may be recessed relative to adjacent exterior surfaces of the shroud 506 and the housing portion 508, as shown in FIG. 5C.

In some embodiments of the present technology, the shroud 506 may form part of a capacitive touch detector. More specifically, the shroud 506 may form a dielectric portion of a capacitor of the capacitive touch detector. The shroud 506 may have a generally conical or generally cylindrical outer surface, which provides a relatively large touchable surface area for touching by the user's finger(s), compared with a touchable surface area of the gasket 410 described above. The shroud adaptor 514, which is in contact with and supports at least one internal surface of the shroud 506, may be formed of a conductive material (e.g., metal), and may serve as an inner conductor of the capacitor. The shroud adaptor 514 may be electrically connected to sensing circuitry of the capacitive touch sensor via at least one pin 512 (e.g., a pogo pin), which may extend from a printed circuit board or other structure in the housing, thus connecting the shroud adaptor 514 to circuitry on the printed circuit board. The pin(s) 512 may, e.g., apply a known charge to the shroud adaptor 514 when the ultrasound device is powered on. As will be appreciated, a non-conductive grounded material may separate the shroud 506 from the lens, so that operation of the capacitive touch detector does not affect the ultrasonic waves transmitted and/or received through the lens.

Similar to the descriptions above, when the touchable surface area of the shroud 506 is not being touched by a finger, the sensing circuitry of the capacitive touch sensor may measure a parasitic capacitance, which may be used as a baseline capacitance. However, when the user's finger(s) touch(es) the touchable surface area of the shroud 506, the finger(s) may have sufficient conductivity to serve as an outer conductor of the capacitor and cause a change in capacitance to be measured by the sensing circuitry. The sensing circuitry may be sufficiently sensitive that a touch by a gloved finger may have sufficient conductivity to cause a measurable change in capacitance. Advantageously, the shroud 506 enables a touch as well as consecutive touches to be detectable at any location on its 360° circumferential touchable surface, thus allowing the user to use any available finger(s) to provide touch input to a processing device (e.g., 104).

In some embodiments of the present technology, touch detection circuitry may be provided in an ultrasound device to enable detection of not only a single touch or consecutive single touches (e.g., by one finger) on a capacitive touch surface, but may also enable detection of multiple simultaneous touches at multiple different touch spots on the capacitive touch surface. For example, the capacitive touch surface may be located on a shroud (e.g., 506) of the ultrasound device or on a housing portion (e.g., 508) near the shroud. Discrete sensing elements may be provided at a plurality of different locations below the capacitive touch surface. A finger touch on a portion of the capacitive touch surface above one of the sensing elements may be sensed by one or more of the sensing elements below the touched portion but not by others of the sensing elements. When simultaneous touches are detected at multiple different portions of the capacitive touch surface, it may be determined by the touch detection circuitry or by a processing device operatively connected to the touch detection circuitry that a multi-touch event occurred, and a control operation of the ultrasound device may be initiated according to the detected multi-touch event.

The multiple discrete sensing elements of the capacitive touch surface may be produced using known techniques for producing, e.g., touch-screen displays, computer touch pads, and the like. For example, the capacitive touch surface may be produced by known techniques for forming multilayer patterned films of conductive layers and dielectric layers, in which a first conductive layer is patterned to produce a plurality of conductive rows, and in which a second conductive layer is patterned to produce a plurality of parallel conductive columns, which may be orthogonal to the parallel conductive rows and arranged above the parallel conductive rows. A first dielectric layer may separate the parallel conductive columns from the parallel conductive rows. A second dielectric layer may be arranged above the parallel conductive columns and may serve as an external surface that may be touched by the user. Known circuitry may be used to connect to each of the rows and columns individually, and to address each individual row and each individual column. Using known techniques, a location of a touch may be determined by a location of capacitance change at an intersection of a row and a column below the touch, and, similarly, locations of simultaneous touches may be determined by locations of intersections of rows and columns at which capacitance changes are measured.

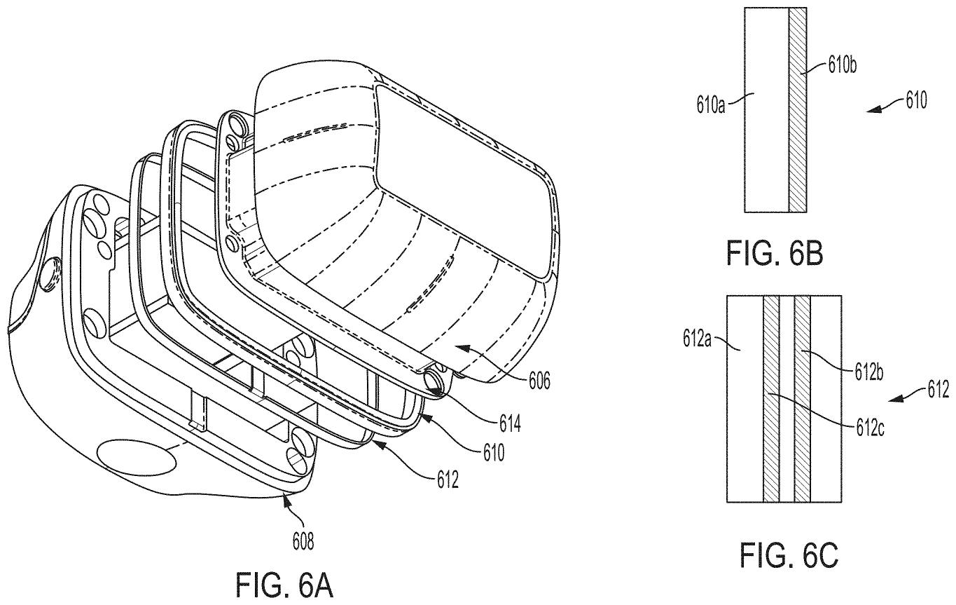

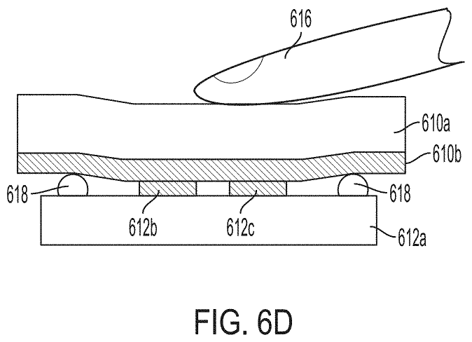

In alternative embodiments of the present technology, instead of detection of a touch via capacitive sensing techniques, a touch may be detected via resistive techniques. FIG. 6A shows a perspective view of a partially disassembled head region of an ultrasound device capable of detecting a resistive change caused by a user's touch, according to some embodiments. The head region includes a shroud 606 attached to a housing portion 608 via a flexible outer ring 610. The shroud 606 may be formed of a non-conductive material (e.g., hard plastic) and may be electrically grounded. The housing portion 608 may be formed of a non-conductive material (e.g., hard plastic) and may be electrically grounded. The shroud 606 may be supported by a shroud adapter 614, which may be contoured to support at least one internal surface of the shroud 606. The shroud adapter 614 may be formed of a hard material, conductive or non-conductive, and may be electrically grounded. The flexible outer ring 610, which may be floating, may comprise an outer layer 610a of non-conductive material and an inner layer 610b of conductive material. The flexible outer ring 610 may completely encircle a circumference of the ultrasound device, or may partially encircle the ultrasound device (e.g., 50% or more; or 70% or more; or 90% or more). FIG. 6B shows a cross section of a portion of the flexible outer ring 610. In some embodiments, the outer layer 610a may be formed of a flexible and resilient plastic (e.g., polyvinyl chloride), which may flex and then return to its original shape. The inner layer 610b may be formed of metal, e.g., aluminum or copper, which may be clad onto the outer layer 610a and resiliently flex with the outer layer 610a. An exterior surface of the flexible outer ring 610 may be flush with adjacent exterior surfaces of the shroud 606 and the housing portion 608.

A shorting strip 612 may be positioned interior to the flexible outer ring 610. FIG. 6C shows a plan view of a section of the shorting strip 612 that faces the inner layer 610b of the flexible outer ring 610. The shorting strip 612 may comprise a non-conductive ring 612a supporting parallel metallic rings 612b, 612c. The metallic rings 612b, 612c may be electrically isolated from each other, and each of the metallic rings 612b, 612c may be electrically connected to sensing circuitry of a resistive touch sensor, which may, e.g., apply a known charge to one or both of the metallic rings 612b, 612c. In some embodiments, metallic rings 612b, 612c may be electrically connected to circuitry on a printed circuit board or other structure in the housing. FIG. 6D schematically shows a cross-sectional view of the metallic rings 612b, 612c of the shorting strip 612 being shorted by the inner layer 610b of the flexible outer ring 610 under weight of a finger 616. Non-conductive spacers 618 on the shorting strip 612 and/or on flexible outer ring 610 may separate the inner layer 610b of the flexible outer ring 610 from the metallic rings 612b, 612c of the shorting strip 612 when no touching is occurring on the flexible outer ring 610.

When the exterior surface of the flexible outer ring 610 is not being touched by a finger, the sensing circuitry of the resistive touch sensor may measure an open circuit or a resistance that is above a predetermine threshold, which may be used as a baseline resistance. However, when the user's finger touches the exterior surface of the flexible outer ring 610, due to the flexibility of the flexible outer ring 610 the conductive inner layer 610b may bend to contact the metallic rings 612b, 612c of the shorting strip 612, causing the metallic rings 612b, 612c to be electrically shorted with each other. Thus, the touch may be detected by the sensing circuitry as a change in resistance (e.g., a short). Advantageously, touches may be detectable by the sensing circuitry even if the user is wearing thick gloves. Also advantageously, the flexibility of the flexible outer ring 610 is such that even gentle touches may be sufficient to cause a short to occur in the shorting strip 612. That is, a touch may be detected in which little or no force is applied (e.g., less than about 75 mg, less than about 50 mg, of less than about 25 mg). For example, a finger already touching an external surface of the ultrasound device may be gently slid onto the flexible outer ring 610, and the weight of the finger may be sufficient to cause movement of the flexible outer ring 610 so that the inner layer 610b contacts and shorts together the metallic rings 612b, 612c. Although FIGS. 6A-6D show an implementation where shorting occurs by the inner layer 610b making contact with one type of structure, it should be understood that other structures may be used for shorting instead of the metallic rings 612b, 612c (e.g., a pair of conductive pads, a plurality of pairs of conductive pads, a ring of conductive pads, etc.).

As will be appreciated, with the embodiments described above touch(es) may be detected by the touch detection circuitry (e.g., 124) during an ultrasound imaging session without adversely affecting data being obtained during the ultrasound imaging session. The touch detection circuitry may send a signal of the touch(es) to a processing device (e.g., 104), to cause the processing device to control a parameter or a characteristic of the ultrasound imaging session. In some embodiments of the present technology, the touch detection circuitry may be powered on when the ultrasound device is powered on, such that touch(es) may be detected before and/or after an ultrasound imaging session.

Referring now to the processing device 104, the processor 114 may include specially programmed and/or special-purpose hardware such as an application-specific integrated circuit (ASIC). The processing device 104 may be configured to process ultrasound data received from the ultrasound device 102 to generate ultrasound images for display on the display screen 112. The processing may be performed by, for example, the processor 114. The processor 114 may also be adapted to control acquisition of ultrasound data with the ultrasound device 102. The ultrasound data may be processed in real-time during a scanning session, while the echo signals are being received. For example, ultrasound data may be acquired even as images are being generated based on previously acquired data and while a live ultrasound image is being displayed. As additional ultrasound data is acquired, additional frames or images generated from more-recently acquired ultrasound data may be sequentially displayed. Additionally, or alternatively, the ultrasound data may be stored temporarily in a buffer during a scanning session and processed in less than real-time. In some embodiments of the present technology, the displayed ultrasound image may be updated at a rate of at least 5 Hz, at least 10 Hz, at least 20 Hz, at a rate between 5 and 60 Hz, at a rate of more than 20 Hz, which may be changed based on a user touch.

The processing device 104 may be configured to perform processes, including those described below and elsewhere herein, using the processor 114 (e.g., one or more computer hardware processors) and one or more articles of manufacture, which may include non-transitory computer-readable storage media (e.g., the memory 116). The processor 114 may control writing data to and reading data from the memory 116 in any suitable manner. To perform certain of the processes described herein, the processor 114 may execute one or more processor-executable instructions stored in one or more non-transitory computer-readable storage media (e.g., the memory 116), which may serve as non-transitory computer-readable storage media storing processor-executable instructions for execution by the processor 114. The camera 120 may be configured to detect light (e.g., visible light) to form an image. The camera 120 may be on a same face of the processing device 104 as the display screen 112. The display screen 112 may be configured to display images and/or videos, and may be, for example, a liquid crystal display (LCD), a plasma display, and/or an organic light emitting diode (OLED) display on the processing device 104. The input device 118 may include one or more devices capable of receiving input from a user and transmitting the input to the processor 114. For example, the input device 118 may include a keyboard, a mouse, a microphone, touch-enabled sensors on the display screen 112, and/or a microphone. The display screen 112, the input device 118, the camera 120, and the speaker 122 may be communicatively coupled to the processor 114 and/or under the control of the processor 114.

It should be appreciated that the processing device 104 may be implemented in any of a variety of ways. For example, the processing device 104 may be implemented as a handheld device such as a mobile smartphone or a tablet. Thereby, a user of the ultrasound device 102 may be able to operate the ultrasound device 102 with one hand and hold the processing device 104 with another hand. In other examples, the processing device 104 may be implemented as a portable device that is not a handheld device, such as a laptop. In yet other examples, the processing device 104 may be implemented as a stationary device such as a desktop computer. The processing device 104 may be connected to the network 106 over a wired connection (e.g., via an Ethernet cable) and/or a wireless connection (e.g., over a WiFi network). The processing device 104 may thereby communicate with (e.g., transmit data to or receive data from) the one or more servers 108 over the network 106. For example, a party may provide from the server 108 to the processing device 104 processor-executable instructions for storing in one or more non-transitory computer-readable storage media which, when executed, may cause the processing device 104 to perform certain of the processes described herein. For further descriptions of ultrasound devices and systems, see U.S. patent application Ser. No. 15/415,434 titled “UNIVERSAL ULTRASOUND DEVICE AND RELATED APPARATUS AND METHODS,” filed on Jan. 25, 2017 and published as U.S. Pat. App. Publication No. 2017-0360397 A1 (and assigned to the assignee of the present application), the contents of which is incorporated by reference herein.

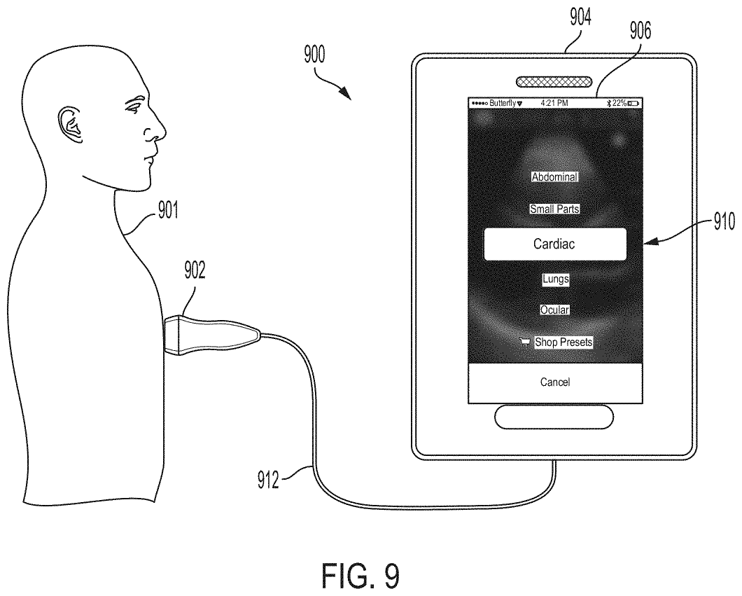

FIG. 9 is a schematic diagram illustrating how an ultrasound device may be used to image a subject, in accordance with some embodiments of the technology described herein. In particular, FIG. 9 shows an ultrasound system 900 comprising an ultrasound device 902 communicatively coupled to processing device 904 via a communication link 912. The ultrasound device 902 may be used to image a subject 901. Aspects of an imaging procedure (e.g., an imaging mode, a gain, start/stop of recording, freezing a displayed image, etc.) may be selected by a touch or touches on the ultrasound device 902, according to the technology described herein, or by a selection made on a graphical user interface 910 displayed on a display 906 of the processing device 904. Although the processing device 904 is shown to be a tablet computer, as mentioned elsewhere herein the processing device 904 have other forms, including but not limited to: a smart phone, a laptop computer, and the like. Further, although, the communication link 912 is shown to be a wired link, as mentioned elsewhere herein other forms of communication may be used, including but not limited to: a Bluetooth, a Wi-Fi connection, and the like. Advantageously, because the ultrasound device 902 may be held in one hand of the user, and because touched-based input may be made on the ultrasound device 902 with the same hand as that holding the ultrasound device 902, the user's other hand is free to hold, e.g., the table computer.

FIG. 1 as well as the other figures should be understood to be non-limiting. For example, the ultrasound system 100 may include fewer or more components than shown and the processing device 104 and ultrasound device 102 may include fewer or more components than shown. In some embodiments, the processing device 104 may be part of the ultrasound device 102.

In some embodiments, the processing device 104 may be configured to perform one or more actions based on receiving, from the ultrasound device 102, an indication of the detection of one or more touches by the touch detection circuitry 124 during ultrasound imaging (after an ultrasound imaging session has begun, after the ultrasound device 102 has begun to collect ultrasound data, and/or while the ultrasound device 102 is placed on a subject or patient for ultrasound imaging). The touch circuitry 124 may be configured to detect touches having little or no force, so that there is no adverse interference with the ultrasound device's imaging (as described above). In response to the detected touch(es), the processing device 104 may control aspects of the ultrasound imaging, and not just initiation of ultrasound imaging. In some embodiments, the aspects controlled by the processing device 104 may include any one or any combination of: an initiation of recording of a cine (i.e., a sequence of ultrasound images); a stop to recording of a cine that was previously initiated; a freeze to a current ultrasound image on the display screen 112 of the processing device 104; a saving operation to store in the memory 116 the ultrasound image that is frozen on the display screen 112; a modification of an imaging depth (e.g., a switch between a shallow imaging depth and a deep imaging depth, where the shallow and deep imaging depths may be predetermined for a given imaging preset (i.e., a set of imaging parameters optimized for imaging particular anatomy)); a modification of a gain (e.g., there may be a predetermined sequence of gains, and a touch may signal a desire of the user to advance one gain in the sequence); to toggle an parameter on or off (e.g., to turn on/off imaging modes such as, biplane, spectra Doppler, power Doppler, color Doppler); proceeding to a next mode in a predetermined sequence of imaging modes; a switch between imaging presets; etc. Further, in response to a detected touch, the processing device 104 may modify its own configuration, modifying a configuration of the ultrasound device 102, or both.

In some embodiments of the present technology, the processing device 104 may be configured to provide a user with options for actions that the processing device 104 may be configured to perform based on receiving the indication of the detection of the one or more touches from the ultrasound device 102. In some embodiments, the processing device 104 may be configured to provide these options upon detecting connection of the ultrasound device 102 to the processing device 104 for the first time. In response to receiving a user selection of an action option, the processing device 104 may be configured to configure itself to perform the action based on receiving the indication of the detection of the one or more touches from the ultrasound device 102. In some embodiments, if the user does not select an action option, the processing device 104 may be configured to select a default action option (e.g., capturing a cine).

In some embodiments of the present technology, the processing device 104 may be configured to provide a user with options for timing (e.g., a length of a quiet-time window and a length of a latency time window) that the ultrasound device 102 may use to detect sequential touches. In some embodiments, the processing device 104 may be configured to provide these options upon detecting connection of the ultrasound device 102 to the processing device 104 for the first time. In some embodiments, if the user does not select a timing option, the processing device 104 may be configured to select a default timing option (e.g., a certain length of time for the quite time window and the latency time window).

In some embodiments of the present technology, the processing device 104 may be configured to provide a user with options for a time duration that detection of one or more touches causes an action to be performed. For example, the duration may include a certain number of minutes (e.g., 5, 15, or 30 minutes) after the user activates touch functionality, a length of a study performed after the user activates on the touch functionality, and/or until the user turns off the touch functionality. In some embodiments, the processing device 104 may be configured to provide these options upon detecting connection of the ultrasound device 102 to the processing device 104 for the first time. In some embodiments, if the user does not select a duration option, the processing device 104 may be configured to select a default duration option (e.g., a certain number of minutes, such as 15).

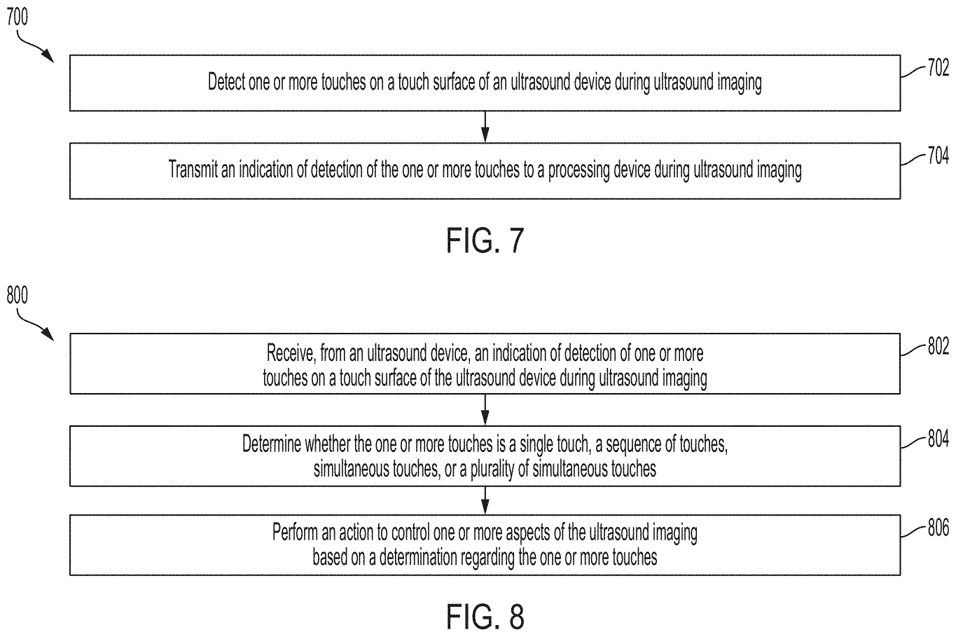

FIG. 7 shows a process 700 for detecting one or more touches on an ultrasound device (e.g., 102) during ultrasound imaging, in accordance with some embodiments of the present technology. The process 700 may be performed by the ultrasound device. In act 702, the ultrasound device may detect one or more touches on an exterior touch surface of the ultrasound device during ultrasound imaging (e.g., after the ultrasound device has begun to collect ultrasound data, and/or while the ultrasound device is placed on the subject for ultrasound imaging). In some embodiments, the ultrasound device may be configured to detect a single touch. In some embodiments, the ultrasound device may be configured to detect a sequence of touches. In some embodiments, the ultrasound device may be configured to detect simultaneous touches (e.g., by two or more fingers concurrently). In some embodiments, the ultrasound device may be configured to detect a sequence of simultaneous touches (e.g., consecutive simultaneous touches). In some embodiments, the ultrasound device may be configured to detect and differentiate between a single, a sequence of touches, simultaneous touches, a sequence of simultaneous touches. The ultrasound device may use capacitive touch detection or resistive touch detection circuitry, such as those of the embodiments described above, to detect the touch(es). The touch detection circuitry may be configured to detect touch(es) made with little or no force on the touch surface of the ultrasound device, and thus would not interfere with imaging being performed by the ultrasound device. That is, touch(es) may be detected in which little or no force is applied (e.g., less than about 75 mg, less than about 50 mg, less than about 25 mg, or substantially 0 mg). For example, touch(es) may be detected for a finger or fingers already touching an external surface of the ultrasound device and gently slid onto the touch surface without application of pressure, or with only minimal pressure being applied. The process 700 proceeds from act 702 to act 704.

In act 704, the ultrasound device may transmit an indication of the detection of the one or more touches to a processing device (e.g., 104) during the ultrasound imaging. The processing device may be in operative communication with the ultrasound device. The indication transmitted to the processing device during the ultrasound imaging may include, e.g., an interrupt signal. In some embodiments, the transmitted indication may include an indication of whether a single touch, a sequence or touches, simultaneous touches, or a sequence of simultaneous touches was detected. In response to receiving the indication of the one or more touches, the processing device may perform one or actions

FIG. 8. shows a process 800 for controlling one or more aspects of ultrasound imaging in response to a detection of one or more touches on an ultrasound device (e.g., 102) during the ultrasound imaging, according to some embodiments of the present technology. The process 800 may be performed by a processing device (e.g., 104) in operative communication with the ultrasound device.

In act 802, the processing device may receive from the ultrasound device an indication of detection of one or more touches on an exterior touch surface of the ultrasound device during ultrasound imaging (e.g., after the ultrasound device has begun to collect ultrasound data, and/or while the ultrasound device is placed on the subject for ultrasound imaging). Optionally, in act 804, the processing device may determine whether the received indication is for a single touch, a sequence of touches, simultaneous touches, or a sequence of simultaneous touches, unless such information is provided in the received indication. In act 806, the processing device performs one or more actions based on a determination regarding the one or more touches, to control one or more aspects of the ultrasound imaging (and not just initiation of ultrasound imaging). The aspect(s) controlled by the processing device may include any one or any combination of: an initiation of recording of a cine (i.e., a sequence of ultrasound images); a stop to recording of a cine that was previously initiated; a freeze to a current ultrasound image on the display screen 112 of the processing device 104; a saving operation to store in the memory 116 the ultrasound image that is frozen on the display screen 112; a modification of an imaging depth (e.g., a switch between a shallow imaging depth and a deep imaging depth, where the shallow and deep imaging depths may be predetermined for a given imaging preset (i.e., a set of imaging parameters optimized for imaging particular anatomy)); a modification of a gain (e.g., there may be a predetermined sequence of gains, and a touch may signal a desire of the user to advance one gain in the sequence); to toggle an parameter on or off (e.g., to turn on/off imaging modes such as, biplane, spectra Doppler, power Doppler, color Doppler); proceeding to a next mode in a predetermined sequence of imaging modes; a switch between imaging presets; etc. Further, in response to a detected touch, the processing device 104 may modify its own configuration, modifying a configuration of the ultrasound device 102, or both.

Various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An ultrasound device, comprising:
a housing; and
touch detection circuitry housed at least partially in the housing, the touch detection circuitry being configured to:
detect at least one touch of a user on a circumferential region of the housing during ultrasound imaging, the circumferential region partially or completely encircling an exterior portion of the housing, and
transmit an indication of detection of the at least one touch to a processing device during the ultrasound imaging.

2. The ultrasound device according to claim 1, wherein the touch detection circuitry comprises a touch-sensitive surface on the circumferential region of the housing.

3. The ultrasound device according to claim 2, wherein the touch-sensitive surface is a ring-shaped surface encircling a portion of the housing between a shroud portion that connects a lens portion, through which ultrasonic waves are transmitted and received, and a handle portion configured to be held in a hand of the user.

4. The ultrasound device according to claim 2, wherein the touch-sensitive surface comprises an exterior surface of a shroud portion that connects a lens portion, through which ultrasonic waves are transmitted and received, and a handle portion configured to be held in a hand of the user.

5. The ultrasound device according to claim 1, wherein the touch detection circuitry comprises resistive sensing circuitry for at least one resistive sensor, the resistive sensing circuitry being configured to detect the at least one touch by detecting a change in resistance of at least one part of the circumferential region of the housing.

6. The ultrasound device according to claim 5, wherein the change in resistance is a change from a resistive state when the circumferential region of the housing is not being touched to a short-circuited state when the circumferential region of the housing is being touched.

7. The ultrasound device according to claim 1, wherein the touch detection circuitry comprises capacitive sensing circuitry for at least one capacitive sensor, the capacitive sensing circuitry being configured to detect the at least one touch by detecting a change in capacitance of at least one part of the circumferential region of the housing.

8. The ultrasound device according to claim 1, wherein the touch detection circuitry is configured to detect a plurality of simultaneous touches at a plurality of parts of the circumferential region of the housing.

9. The ultrasound device according to claim 1, wherein the touch detection circuitry is configured to detect a plurality of consecutive touches on the circumferential region of the housing.

10. The ultrasound device according to claim 1, wherein the touch detection circuitry is configured to:
detect at least one touch of a user on the circumferential region of the housing before the ultrasound imaging starts and after the ultrasound imaging ends, and
transmit an indication of detection of the at least one touch to the processing device before the ultrasound imaging starts and after the ultrasound imaging ends.

11. The ultrasound device according to claim 1, wherein
the ultrasound device further comprises the processing device, and
the processing device is external to the housing or is housed at least partially in the housing.

12. The ultrasound device according to claim 11, wherein the processing device is configured to:
receive, from the touch detection circuitry, the indication of detection of the at least one touch, and
perform an action to control an aspect of the ultrasound imaging based on the at least one touch.

13. The ultrasound device according to claim 12, wherein, when performing the action to control the aspect of the ultrasound imaging, the processor controls any one or any combination of:
to record a cine,
to freeze a current ultrasound image on a display screen of the processing device,
to save to memory an ultrasound image that is frozen on the display screen,
to modify an imaging depth,
to modify a gain,
to toggle color Doppler imaging on or off,
to switch imaging modes, and
to switch imaging presets.

14. A method, comprising:
detecting, using touch detection circuitry of an ultrasound device, at least one touch of a user on a circumferential region of the ultrasound device during ultrasound imaging, the circumferential region partially or completely encircling an exterior portion of the ultrasound device; and
transmitting an indication of detection of the at least one touch to a processing device during the ultrasound imaging.

15. The method of claim 14, wherein the detecting detects the at least one touch on a ring-shaped surface encircling a portion of the ultrasound device.

16. The method of claim 14, wherein the detecting detects the at least one touch on a shroud surface encircling a portion of the ultrasound device.

17. The method of claim 14, wherein the detecting detects the at least one touch by detecting a change in resistance of at least one part of the circumferential region.

18. The method of claim 17, wherein the change in resistance is a change from a resistive state, when the circumferential region is not being touched, to a short-circuited state, when the at least one part of the circumferential region is being touched.

19. The method of claim 14, wherein the detecting detects the at least one touch by detecting a change in capacitance of at least one part of the circumferential region.

20. The method of claim 14, wherein the transmitting of the indication of detection of the at least one touch to the processing device causes the processing device to perform an action to control any one or any combination of:
to record a cine,
to freeze a current ultrasound image on a display screen of the processing device,
to save to memory an ultrasound image that is frozen on the display screen,
to modify an imaging depth,
to modify a gain,
to toggle color Doppler imaging on or off,
to switch imaging modes, and
to switch imaging presets.
